SEQUENCE LISTING (36 sequences)

SEQ ID NO: 1 - protein, 1150 aa, Homo sapiens
MAPPLRPLARLRPPGMLLRALLLLLLLSPLPGVWCFSELSFVKEPQDVTVTRKDPVVLDCQAHGEVPIKVTWLKNGAKMSENKRIEVLSNGSLYISEVEGRRGEQSDEGFYQCLAMNKYGAILSQKAHLALSTISAFEVQPISTEVHEGGVARFACKISSHPPAVITWEFNRTTLPMTMDRITALPTGVLQIYDVSQRDSGNYRCIAATVAHRRKSMEASLTVIPAKESKSFHTPTIIAGPQNITTSLHQTVVLECMATGNPKPIISWSRLDHKSIDVFNTRVLGNGNLMISDVRLQHAGVYVCRATTPGTRNFTVAMATLTVLAPPSFVEWPESLTRPRAGTARFVCQAEGIPSPKMSWLKNGRKIHSNGRIKMYNSKLVINQIIPEDDAIYQCMAENSQGSILSRARLTVVMSEDRPSAPYNVHAETMSSSAILLAWERPLYNSDKVIAYSVHYMKAEGLNNEEYQVVIGNDTTHYIIDDLEPASNYTFYIVAYMPMGASQMSDHVTQNTLEDVPLRPPEISLTSRSPTDILISWLPIPAKYRRGQVVLYRLSFRLSTENSIQVLELPGTTHEYLLEGLKPDSVYLVRITAATRVGLGESSVWTSHRTPKATSVKAPKSPELHLEPLNCTTISVRWQQDVEDTAAIQGYKLYYKEEGQQENGPIFLDTKDLLYTLSGLDPRRKYHVRLLAYNNIDDGYQADQTVSTPGCVSVRDRMVPPPPPPHHLYAKANTSSSIFLHWRRPAFTAAQIINYTIRCNPVGLQNASLVLYLQTSETHMLVQGLEPNTKYEFAVRLHVDQLSSPWSPVVYHSTLPEAPAGPPVGVKVTLIEDDTALVSWKPPDGPETVVTRYTILYASRKAWIAGEWQVLHREGAITMALLENLVAGNVYIVKISASNEVGEGPFSNSVELAVLPKETSESNQRPKRLDSADAKVYSGYYHLDQKSMTGIAVGVGIALTCILICVLILIYRSKARKSSASKTAQNGTQQLPRTSASLASGNEVGKNLEGAVGNEESLMPMIMPNSFIDAKGGTDLIINSYGPIIKNNSKKKWFFFQDSKKIQVEQPQRRFTPAVCFYQPGTTVLISDEDSPSSPGQTTSFSRPFGVAADTEHSANSEGSHETGDSGRFSHESNDEIHLSSVISTTPPNL

SEQ ID NO: 2 - protein, 1191 aa, M. musculus
MAPPVRPGMLPLLLLLLLPPLGSVPGVWSFSELFFMKEPQDATVTRKDPVVLDCQAHGEGPIKVTWLKNGAKLSENKRIQVLSNGSLYISEVEGRRGEQSDEGYYQCLAVNKNGAILSQKAHLTLSTISAFEVHPVSTEVHEGGVARFSCKISSTPPAVITWEFNRTALPTTMDRVTALPSGVLQIYDVGPEDAGNYRCVAATIAHKRKSMEASLTIVAANETRSFYMPTIIASPQNVTASLHQTVVLECMATGYPRPIISWSRLDHKSIDVFNTRVLGNGNLIISDVKLQHAGVYVCRATTPGTRNFTVAMATLTVLAPPSFVEWPESLTRPRAGTARFVCQAEGIPSPKMSWLKNGRRIHSNGRIKMYNSKLVINQIIPEDDAIYQCMAENSQGSVLSRARLTVVMSEDRPSAPYNVHAETMSSSAILLAWERPLYNSDKVIAYSVHYMKAEGLNNEEYQVVLGNDTTHYIIDDLEPDSNYTFYIVAYMPLGASQMSDHVTQNTLEDVPLRPPEISLTSRSPTDILVSWLPIPAKYRRGQVVLYRLSFRLSTENAIQVVELPGTVHEYLLEGLKPDSVYLVRITAATRVGLGESSVWTSHRTPKATSVKAPKSPELHLEPLNCTTISVRWLQDTEDPAAIRGYKLFYKEEGQQEHGPIFLDTGDLLYTLSGLDPRRKYHVRLLAYNNMEEGYQADQTVSTPGCVSVRDRMVPPPPPPHHLYAKANTSSSIFLHWRRPAFTTAQVINYTIRCNPVGLQNASLVLYLQTSETHMLVQGLEPNTKYEFAVRLHVDQLSSPWSPVVYHSTLPEAPTGPPVGVKVTLIEDDTALVSWKPPDGPETVVTRYTILYASRKAWIAGEWQVLHREGAITMALLENLVAGNVYIVKISASNEVGEGPFSNSVELAVLPKDASESNQRPKRLDSSNAKVYSGYYHLDQKSMTGIAVGVGIALTCILICVLILIYRSKARKSSASKTAQSGTQPLSQASASVAAGSDMGKNLERATETAESLVPMMPSSFIDAKGGTDLIINSYGPIIKNNTKKKWLFFQDTKKIKVEQTQRRFTQAVCFYQPGTTVLISDEDSPGSPGQTASFPRPFGATALDTEHSANSEGSHETGDSGRFSHESNDEIHLSSVISSTPPTSNPLAGGDSDGDAAPKKHGDPAQPLPAEQTSAPQTSAGLRYAAEGFPV

SEQ ID NO: 3 - protein, 1193 aa, R. norvegicus
MAPPVRPGMLPLLLLLLLPPLGSVPGVWSFSELFFMKEPQDATVTRKDPVFLDCQAHGEGPIKVTWLKNGAKLSENKRIQVLSNGSLYISEAEGRRGEQSDEGFYQCLAVNKYGAILSQKAHLTLSTISAFEVHPVSTEVPEGGVARFSCKISSTPPAVITWEFNRTALPMTMDSRVTALPSGVLQIYDAGPEDAGKYRCVAATHAHKRKSMEASLTIVPANETRSFYMPTIIASPQNVTASLHQTVVLECMATGYPKPIISWSRLDHKSIDVFNTRVLGNGNLIISDVKLQHAGVYVCRATTPGTRNFTVAMATLTVLAPPSFVEWPESLTRPRAGTARFVCQAEGIPSPKMSWLKNGRRIHSNGRIKMYNSKLVINQIIPEDDAIYQCMAENSQGSVLSRARLTVVMSEDRPSAPYNVHAETMSSSAILLAWERPLYNSDKVIAYSVHYMKAEGLNNEEYQVVLGNDTTHYIIDDLEPDSNYTFYIVAYMPMGASQMSDHVTQNTLEDVPLRPPEISLTSRSPTDILISWLPIPAKYRRGQVVLYRLSFRLSTENSIQVVELPGTVHEYLLEGLEPDSVYLVRITAATRVGLGESSVWTSHRTPKATSVKAPKSPELHLEPLNCTTISVRWLQDTEDPAAIRGYKLFYKEEGQQEHGPIFLDTGDLLYTLSGLDPRRKYHVRLLAYNNLEDGYQADQTVSTPGCVSVRDRMVPPPPPPHHLYAKANTSSSIFLHWRRPAFTTAQVINYTIRCNPVGLQNASLVLYLQTSETHMLVQGLEPNTKYEFAVRLHVDQLSSPWSPVVYHTTLPEAPTGPPVGVKVTLIEDDTALVSWKPPDGPETVVTRYTILYASRKAWIAGEWQVLHREGAITMALLENLVAGNVYIVKISASNEVGEGPFPNSVELAVLPKDASESNQRPKRLDSSDAKVYSGYYHLDQKSMTGIAVGVGIALTCILICVLILIYRSKARKSSASKTTQSGTQPLSRASASVAAGSDMGKNLERATENEESSVPMMPSCFIDAKGGTDLIINSYGPIIKNNPKKKWRFFQDTKKIKVEQTQRRITQTVCFYQPGTTVLISDEDSPSSPGQTTSFPRPFGPTTLDTEHSANSEGSHETGDSGRFSHESNDEIHLSSVISSTPPTSNSLTCGDSDGDAAPKKHGDPAQPLPAEQTSAPQPTPAGLRHAAESVPV

SEQ ID NO: 4 - protein, 19 aa, Artificial Sequence
  conditioned medium (CM)
GRDFYKILGVPRSASIKDI

SEQ ID NO: 5 - DNA, 18 bp, Artificial Sequence
  primer
ctatgctcatcggaacag

SEQ ID NO: 6 - DNA, 18 bp, Artificial Sequence
  primer
gtgacacaggtgtccacg

SEQ ID NO: 7 - DNA, 33 bp, Artificial Sequence
  primer
gcaagcggccgcgcagagcccaaatcttgtgac

SEQ ID NO: 8 - DNA, 30 bp, Artificial Sequence
  primer
gcaatctagactatttacccggagacaggg

SEQ ID NO: 9 - DNA, 20 bp, Artificial Sequence
  primer
accacagtccatgccatcac

SEQ ID NO: 10 - DNA, 20 bp, Artificial Sequence
  primer
tccaccaccctgttgctgta

SEQ ID NO: 11 - DNA, 18 bp, Artificial Sequence
  primer
gcctctacatgaagctcc

SEQ ID NO: 12 - DNA, 18 bp, Artificial Sequence
  primer
ccatgactgtcagccagg

SEQ ID NO: 13 - DNA, 30 bp, Artificial Sequence
  primer
gcaaggatccgctgagagggtaggaccagg

SEQ ID NO: 14 - DNA, 28 bp, Artificial Sequence
  primer
gcaagaattcatagccctgcagcccgtt

SEQ ID NO: 15 - DNA, 20 bp, Artificial Sequence
  primer
gagtgtgggtcttcctggtc

SEQ ID NO: 16 - DNA, 20 bp, Artificial Sequence
  primer
cctggtggagtcacagagta

SEQ ID NO: 17 - DNA, 18 bp, Artificial Sequence
  primer
cccttggattagaggctg

SEQ ID NO: 18 - DNA, 18 bp, Artificial Sequence
  primer
ctggctcatcttctactc

SEQ ID NO: 19 - DNA, 19 bp, Artificial Sequence
  primer
cccccacttcaccacactc

SEQ ID NO: 20 - DNA, 21 bp, Artificial Sequence
  primer
gcatcactgagcttctttccc

SEQ ID NO: 21 - DNA, 18 bp, Artificial Sequence
  primer
ggtttggttcaagaatcg

SEQ ID NO: 22 - DNA, 18 bp, Artificial Sequence
  primer
gctgctttgctttcagtc

SEQ ID NO: 23 - DNA, 20 bp, Artificial Sequence
  primer
tgaagcggaagctgcaaaga

SEQ ID NO: 24 - DNA, 20 bp, Artificial Sequence
  primer
ggcccttcgattagaaaacc

SEQ ID NO: 25 - DNA, 21 bp, Artificial Sequence
  primer
gccaagctgcttcattgatgc

SEQ ID NO: 26 - DNA, 21 bp, Artificial Sequence
  primer
gtctccatgcttcttcggtgc

SEQ ID NO: 27 - DNA, 38 bp, Artificial Sequence
  primer
gcaaactagtcatggccaggaagtcatccgcctctaag

SEQ ID NO: 28 - DNA, 30 bp, Artificial Sequence
  primer
gcaagcggccgcgacgggaacactctcggc

SEQ ID NO: 29 - DNA, 27 bp, Artificial Sequence
  primer
gcaagtcgacaggacgcaacggtcaca

SEQ ID NO: 30 - DNA, 31 bp, Artificial Sequence
  primer
gcaagtcgacctagcctgaataaactttggc

SEQ ID NO: 31 - DNA, 31 bp, Artificial Sequence
  primer
gcaagcggccgccatggcgcctcccgtgcgc

SEQ ID NO: 32 - DNA, 56 bp, Artificial Sequence
  primer
gcaagcggccgctagtaatctggaacatcgtatgggtacttcctggctttgcttcg

SEQ ID NO: 33 - DNA, 31 bp, Artificial Sequence; identical to SEQ ID NO: 31
  primer

SEQ ID NO: 34 - DNA, 30 bp, Artificial Sequence; identical to SEQ ID NO: 28
  primer

SEQ ID NO: 35 - DNA, 20 bp, Artificial Sequence
  primer
ttacttcccgccagctcttc

SEQ ID NO: 36 - DNA, 20 bp, Artificial Sequence
  primer
gcattttgggggtatctctc

(19) United States
(12) Patent Application Publication
(10) Pub. No.: US 2005/0287664 A1
(43) Pub. Date: Dec. 29, 2005

(54) Cellular proliferation control factors

(76) Inventor: Ming-Ji Fann, San-Chung (TW)

Correspondence Address: FISH & RICHARDSON P.C., PO BOX 1022, MINNEAPOLIS, MN, 55440-1022, US

(21) Appl. No.: 11/139,435

(22) Filed: May 27, 2005

Related U.S. Application Data
(60) Provisional application No. 60/575,611, filed on May 27, 2004.

(51) Int. Cl. C12N 5/06; C12N 5/08

(52) U.S. Cl. 435/366; 435/354

(57) ABSTRACT

We discovered that Shen-Dan (SD), and its ligand HEDJ/ERdj3, are important for embryonic stem cell proliferation. These factors also promote neoplastic cell proliferation. Accordingly, compositions comprising SD or HEDJ/ERdj3 are provided, as well as uses thereof.

### RELATED APPLICATIONS

This application claims the benefit of U.S. Provisional Application No. 60/575,611, filed May 27, 2004. The disclosure of the prior application is incorporated herein by reference in its entirety.

### FIELD OF THE INVENTION

This invention relates to factors that control cellular proliferation, particularly embryonic stem cells and neoplastic cells.

### BACKGROUND

Embryonic stem (ES) cells propagated in vitro are essential reagents in the recent progress of mammalian genetics. The ability of these cells to differentiate into a variety of cell types also raises the possibility of using ES cells in regenerative medicine¹⁻⁴. The culture conditions required for the maintenance of pluripotency of ES cells are therefore under intensive study^{5,6}. Although leukemia inhibitory factor (LIF)^{7,8} and its related cytokines are required for culturing mouse ES cells, other factors may also preserve ES cell properties^{9,10}.

### SUMMARY OF THE INVENTION

We identified a receptor, Shen-Dan (SD), on ES cells. We further uncovered HEDJ/ERdj3 as a SD ligand using a yeast two-hybrid screen and by an in situ binding assay. Surprisingly, HEDJ/ERdj3, previously known as a chaperone protein, is a secreted protein and supports proliferation of ES cells and maintains the ES cell phenotype without involvement of the LIF signaling pathway. These results indicate that activation of SD by HEDJ/ERdj3 represents an alternative pathway that can sustain self-renewal of ES cells, and offer a new window into stem cell biology.

Accordingly, one aspect of the present invention provides a method for proliferating embryonic stem cells, comprising contacting at least one embryonic stem (ES) cell with an effective amount of HEDJ/ERdj3 to result in enhanced proliferation of the ES cell. “Enhanced proliferation” means that proliferation is increased by at least about 20%, more preferably by at least about 35%, 50%, 75%, 100%, 150% or 200%. The ES cell may be any ES cell, and preferably a mouse or human ES cell. The HEDJ/ERdj3 can be provided by using any method established in the art, for example by incubating the ES cell with a feeder cell that expresses the HEDJ/ERdj3, or by adding the HEDJ/ERdj3 to the culture medium.

Another aspect of the present invention provides a method of enhancing differentiation of embryonic stem (ES) cells, comprising contacting at least one ES cell with an effective amount of an antagonist for SD. An “antagonist for SD” is a substance that inhibits the function of SD. By way of example, the antagonist may be a compound or protein that inhibits the binding of HEDJ/ERdj3 to SD, SD signal transduction, or transcription or translation of SD. In particular, the antagonist may be an antibody, preferably one that recognizes either SD or HEDJ/ERdj3. The antagonist may also be a molecule that comprises the extracellular domain of SD, or the region of SD containing five fibronectin type III repeats, which mediate the binding between SD and HEDJ/ERdj3. The antagonist may be an analog of HEDJ/ERdj3 that binds to SD but does not trigger the biological activity of SD. In this embodiment, the HEDJ/ERdj3 analog preferably contains a sequence that is at least about 80%, 85%, 90%, 95%, or 98% identical to the sequence of HEDJ/ERdj3.

A further aspect of the present invention provides an isolated antibody that recognizes SD or HEDJ/ERdj3 and inhibits ES cell proliferation. Similarly, also provided is an antibody that recognizes SD or HEDJ/ERdj3 and enhances ES cell proliferation. The antibody may be monoclonal or polyclonal.

Another aspect of the present invention provides a method of screening for agents that enhance ES cell proliferation, comprising:
(a) determining the activity of a test agent to bind to SD and identifying the agents that bind SD as potential agents; and
(b) determining the activity of the potential agents to enhance ES cell proliferation.

Also provided is a method of screening for agents that enhance ES cell differentiation, comprising:
(a) determining the activity of a test agent to inhibit the binding between HEDJ/ERdj3 and SD, and identifying the agents that inhibit as potential agents;
(b) determining the activity of the potential agents to enhance ES cell differentiation.

Yet another aspect of the present invention provides a method of screening for agents that inhibit ES cell proliferation, comprising:
(a) determining the activity of a test agent to inhibit the binding between HEDJ/ERdj3 and SD, and identifying the agents that inhibit as potential agents;
(b) determining the activity of the potential agents to inhibit ES cell proliferation.

The effect of SD is not limited to ES cells. For example, we discovered that SD is expressed in breast cancer cells, and over-expression of SD in fibroblasts led to foci formation. Therefore, it is contemplated that SD is a general proliferative factor, or oncogenic protein. Over-expression of SD, or an SD mutation that results in increased activity, can lead to tumor formation. Accordingly, the present invention provides a method of enhancing proliferation of a cell, comprising contacting the cell with an effective amount of SD or HEDJ/ERdj3 to enhance proliferation of the cell.

The present invention further provides a method of inhibiting cellular proliferation, comprising contacting a cell with an effective amount of an antagonist of SD to inhibit proliferation of the cell. The cell is preferably a neoplastic cell. A “neoplastic cell” is a cell that proliferates without the normal growth inhibition properties. A new growth comprising neoplastic cells is a neoplasm, or tumor. A neoplasm is an abnormal tissue growth, generally forming a distinct mass, which grows by cellular proliferation more rapidly than normal tissue growth. Neoplasms may show partial or total lack of structural organization and functional coordination with normal tissue. As used herein, a neoplasm is intended to encompass hematopoietic neoplasms as well as solid neoplasms. A neoplasm may be benign (benign tumor) or malignant (malignant tumor or cancer). Malignant tumors can be broadly classified into three major types. Malignant neoplasms arising from epithelial structures are called carcinomas, malignant neoplasms that originate from connective tissues such as muscle, cartilage, fat or bone are called sarcomas and malignant tumors affecting hematopoietic structures (structures pertaining to the formation of blood cells) including components of the immune system, are called leukemias and lymphomas. Other neoplasms include, but are not limited to neurofibromatosis.

The method can be used to inhibit cellular proliferation in vitro or in vivo. When practiced in vivo, the method is preferably used in a subject that suffers from, or is suspected of having, a tumor.

A further aspect of the present invention provides an isolated antibody that recognizes SD or HEDJ/ERdj3 and inhibits cellular proliferation, particularly proliferation of a neoplastic cell. Similarly, also provided is an antibody that recognizes SD or HEDJ/ERdj3 and enhances cellular proliferation. The antibody may be monoclonal or polyclonal.

Also provided by the present invention is a method of screening for agents that enhance cellular proliferation, comprising:
(a) determining the activity of a test agent to bind to SD and identifying the agents that bind SD as potential agents; and
(b) determining the activity of the potential agents to enhance cellular proliferation.

Another aspect provides a method of screening for agents that inhibits cellular proliferation, comprising:
(a) determining the activity of a test agent to inhibit the binding between HEDJ/ERdj3 and SD, and identifying the agents that inhibit as potential agents; and
(b) determining the activity of the potential agents to inhibit proliferation of a cell of interest.
   Thus, HEDJ/ERdj3 and SD, as well as analogs thereof, can serve as therapeutic targets for tumors.

Further provided by the present invention are analogs of SD or HEDJ/ERdj3 that enhances cellular proliferation, particularly ES cell proliferation. The analog preferably contains a sequence that is at least about 50%, 60%, 70%, 80%, 85%, 90%, 95%, or 98% identical to the sequence of SD or HEDJ/ERdj3 from any species.

Moreover, the present invention provides analogs of SD or HEDJ/ERdj3 that inhibit cellular proliferation, particularly neoplastic cell proliferation. The analog preferably contains a sequence that is at least about 50%, 60%, 70%, 80%, 85%, 90%, 95%, or 98% identical to the sequence of SD or HEDJ/ERdj3 from any species. Alternatively, the analog may contain a fragment of SD or HEDJ/ERdj3. The fragment is at least 20, 30, 40, 50, 75, 100, 150, 200, 300, 400 or 500 amino acids in length. Of particular interest are fragments that contain at least 2 or 3 fibronectin type III repeats of SD. More preferably, the fragment contains all 5 fibronectin type III repeats of SD.

Isolated nucleic acids coding for SD or HEDJ/ERdj3, or analogs thereof, are also provided. Further provided are vectors comprising the nucleic acids, host cells comprising the nucleic acids or vectors, as well as methods of expressing the encoded proteins using the nucleic acids, vectors, and/or host cells.

The details of one or more embodiments of the invention are set forth in the disclosure below. Other features, objects, and advantages of the invention will be apparent from the description, drawings, and claims.

### BRIEF DESCRIPTION OF THE DRAWINGS

FIG. 1

Protein structure and expression of sd gene. a, Sequence alignment of the human (H. sapiens; SEQ ID NO:1), mouse (M. musculus; SEQ ID NO:2), and rat (R. norvegicus; SEQ ID NO:3) SD protein. Identical residues are shaded. The signal sequence (SS), immunoglobulin (Ig) domains, fibronectin type III (FnIII) domains, and transmembrane region (Tm) are labeled on the top. Putative N-glycosylation sites are marked with asterisks at the bottom. b, Comparison of the protein structures and chromosomal locations of SD, Nope, Punc, Neogenin and DCC. Percentages of identity in extracellular regions between SD and other proteins are indicated. c, Expression of sd was analyzed by Northern blot. Two transcripts with sizes of 8.7 kb and 4.2 kb can be observed. Ad, adult; Br, brain; Nt, neural tube. d, (Upper Panel) Whole-mount in situ hybridization of sd in rat E9 embryo. A, anterior; P, posterior. Scale bar, 200 μm. (Lower Panel) Section of the embryo at the level marked in the upper panel. Scale bar, 50 μm.

FIG. 2

SD is a cell surface membrane protein in mouse ES cells. a, Total RNAs of cultured cells were reverse transcribed with (+) or without (−) reverse transcriptase (RT) before PCR. Gapdh was used as RNA loading control. b, Cell lysates of HEK293T cells transfected with vector, pSDf (encodes full length SD), pSDet (extracellular and transmembrane region), or pSDc (cytoplasmic tail) were subjected to Western analysis using SD1 and SD2 mAbs, and W4 rabbit serum. Tissue homogenate of E10.5 embryos (E10.5) was also analyzed. The specificity of the antibodies was confirmed by adding peptide E or peptide C (E or C). c, Confocal micrographs showing that SD protein is present on the cell surface of R1 and AB2.2 cells. R1 cultured on MEF, and AB2.2 cultured on STO were labeled with SD2 mAb (subtype IgG1a) and SSEA-1 mAb (subtype IgM) and visualized with subtype specific secondary antibodies. Scale bar, 20 μm. d, Inhibition of alkaline phosphatase activity in R1 and AB2.2 cells cultured on feeder cells for 3 days in the presence of SD1 mAb and SDe-Fc protein (*, p<0.01 by student's t-test).

FIG. 3

HEDJ/ERdj3 binds to the SD extracellular domain. a, Schematic representation of eight HEDJ/ERdj3 clones identified by yeast two-hybrid screening and their interaction with the SD extracellular domain, shown as activity in an X-gal assay. b, Various sd deletion mutants were examined for their ability to interact with C-terminal HEDJ/ERdj3. c, HEK293T cells were transiently transfected with pHEDJ or a control vector. Three days after transfection, conditioned medium (CM) and total cell lysate (TCL) were subjected to Western blotting to detect HEDJ/ERdj3 using anti-myc antibody. d, Quantification of HEDJ/ERdj3 binding to SD-expressing cells. HEK293T cells were transiently transfected with pSDf or control vector. SD expression was detected by W4 antiserum. Binding of HEDJ/ERdj3 on the cells was detected by anti-myc mAb. Cell nuclei were visualized by DAPI. Binding of HEDJ/ERdj3 to SD-expressing cells is partially inhibited by 40 μg/ml SDe-Fc. Data were normalized relative to fluorescence intensity of the cells transfected with pSDf in the presence of control CM. Results are shown as means±s.e.m., from a total of 50 cells. (*, p<0.001; by student's t-test).

FIG. 4

Increase of ES cell proliferation and maintenance of ES cell pluripotency by HEDJ/ERdj3. a, Quantification of numbers of R1 clones grown on non-transfected HEK293T cells (open bars) or HEDJ/ERdj3-overexpressing HEK293T cells (closed bars) in the presence of various competitors. Hatched bars are R1 cells grown on HEK293T cells in the presence of 10 ng/ml LIF. (n=3; *, p<0.001; by student's t-test). b, Size distribution of R1 clones grown on non-transfected cells or HEDJ/ERdj3-overexpressing HEK293T cells. c, Increase of AB2.2 cell proliferation by HEDJ/ERdj3. a, Quantification of numbers of AB2.2 clones grown on HEDJ/ERdj3-overexpressing HEK293T cells in the presence of various competitors. More clones are present when AB2.2 are grown on HEDJ/ERdj3-overexpressing HEK293T cells and this can be blocked by the SD1 mAb and the secreted SDe-Fc protein (n=3; *, p<0.001; by student's t-test). b, Size distribution of AB2.2 clones grown on non-transfected cells or HEDJ/ERdj3-overexpressing HEK293T cells. d, RT-PCR was used to analyze ES cell markers (Nanog, Oct4), various ectoderm markers (Sox1, Pax6, nestin), a mesoderm marker (Brachyury), and an endoderm marker (GATA-4). e, RT-PCR analysis of embryoid bodies (EB) at different days that are derived from HEDJ/ERdj3-cultured AB2.2 cells. f, Hematoxylin and eosin staining of teratoma developed from HEDJ/ERdj3-cultured AB2.2 cells. Scale bar, 50 μm. g, Tyrosine phosphorylation of Stat3 was analyzed by Western blotting after incubation of R1 and AB2.2 cells with 25 μg/ml Fc-tagged HEDJ/ERdj3 or 10 ng/ml LIF for various times.

FIG. 5

Expression of SD in 9 esophageal carcinoma (CE) tissues and 9 cell lines derived from esophageal carcinoma. RNA was prepared from esophageal carcinoma (T) and adjacent normal tissues (N), and reverse-transcribed to cDNA. Quantitative PCR was performed to determine expression level of SD and GAPDH. Relative ratios of SD to GAPDH expression are shown. Higher level of SD expression is observed in three CE tumor parts and 5 CE cell lines.

### DETAILED DESCRIPTION

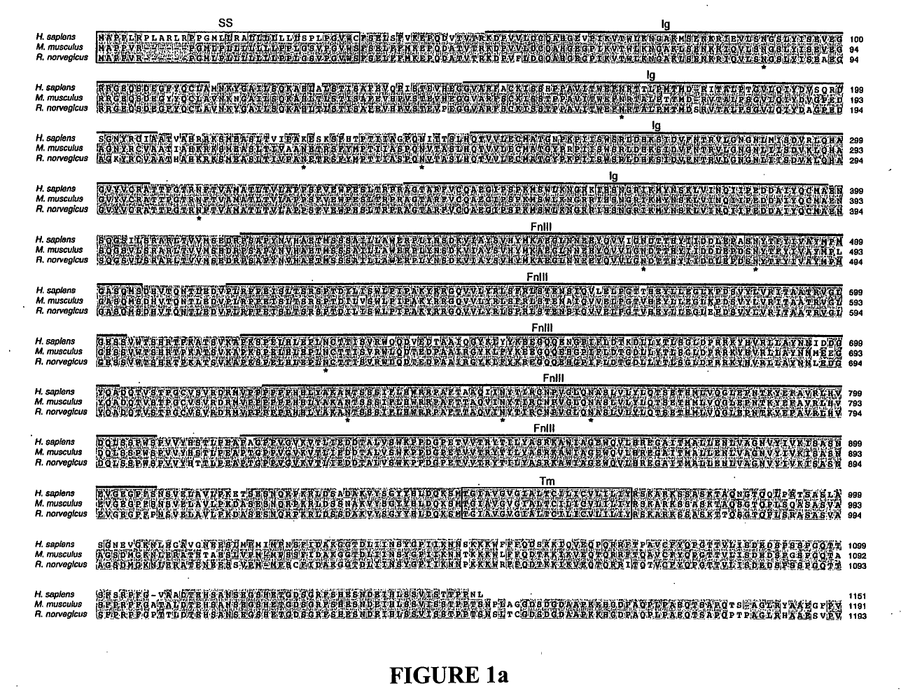

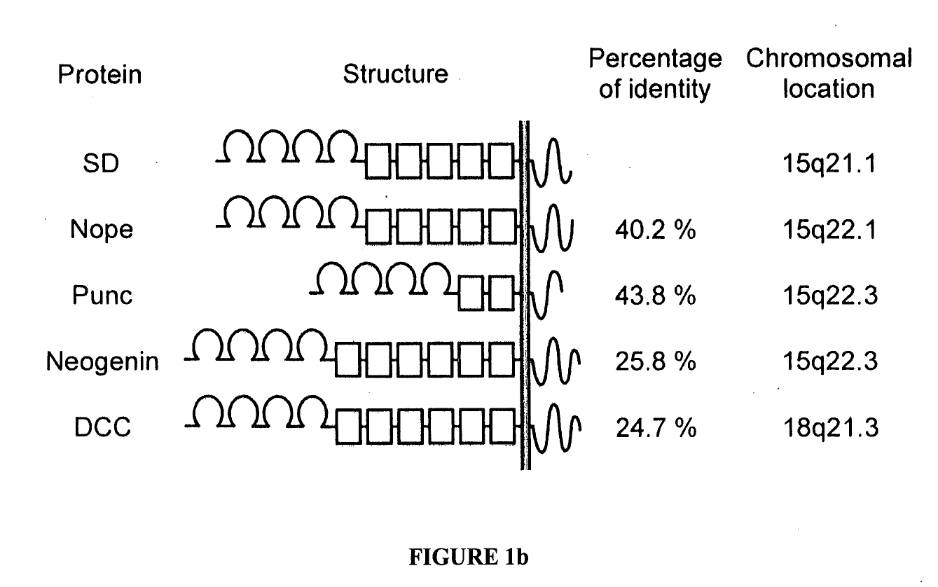

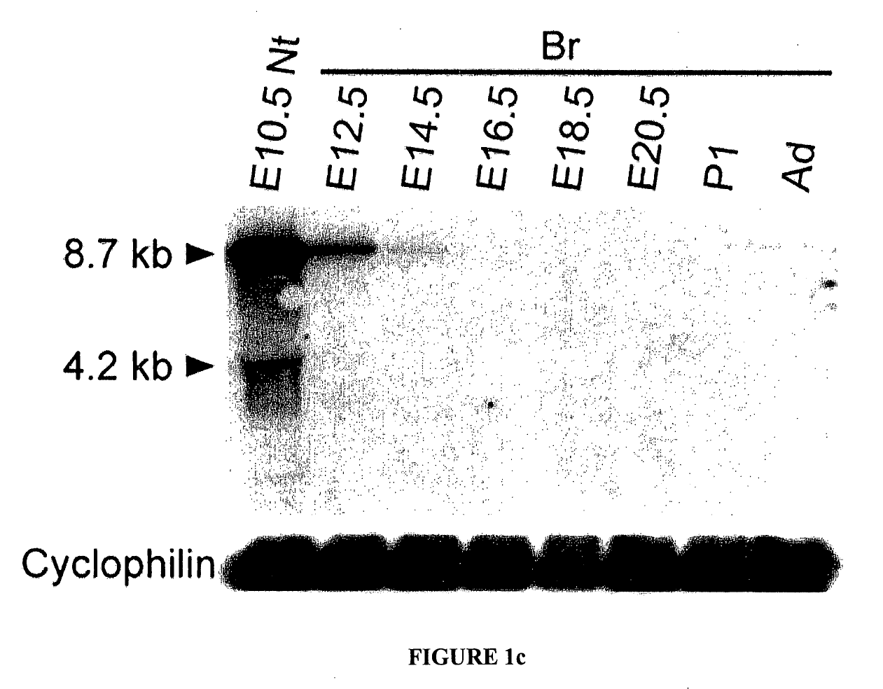

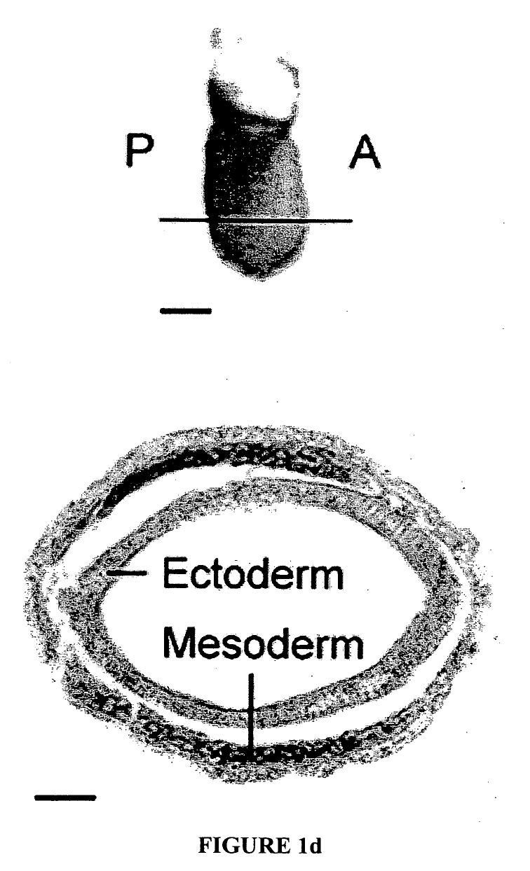

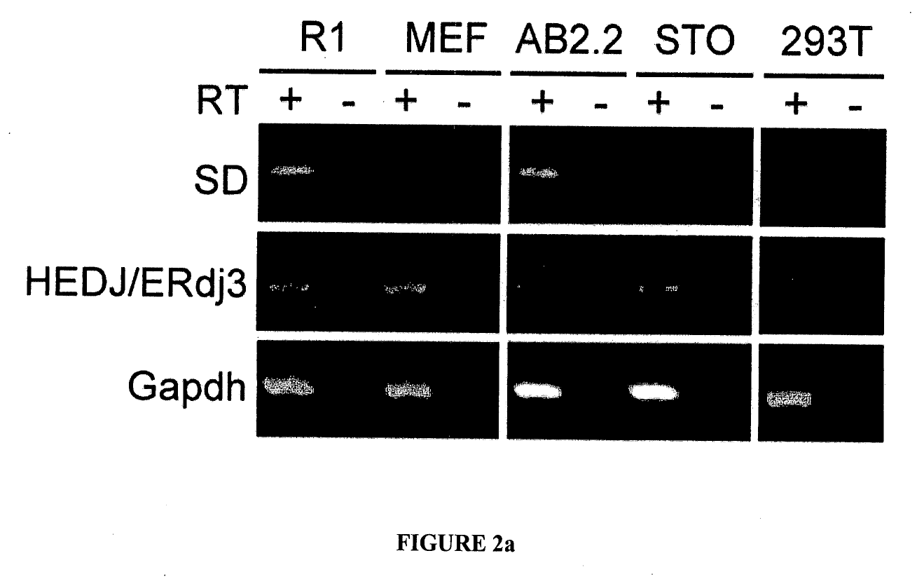

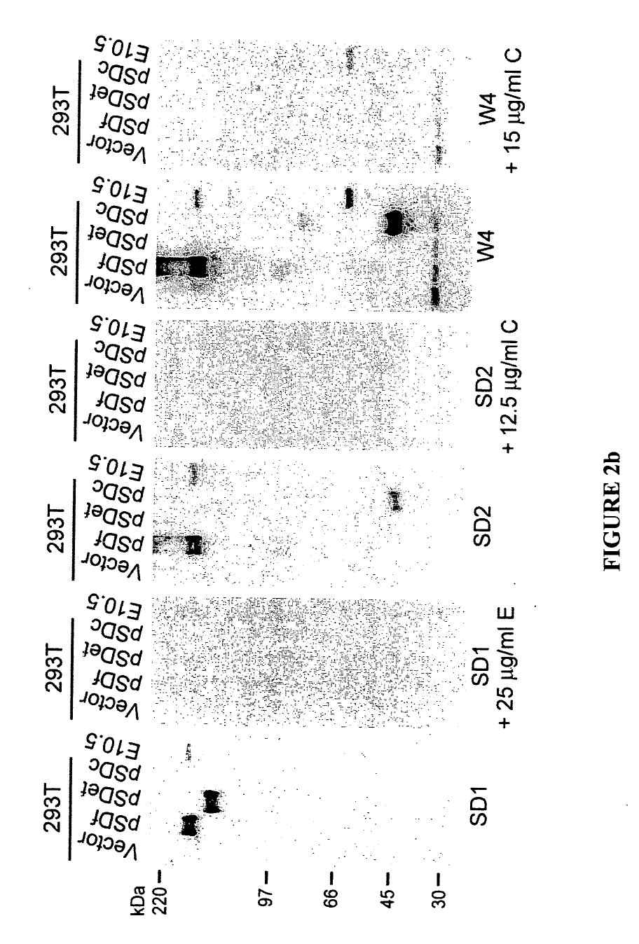

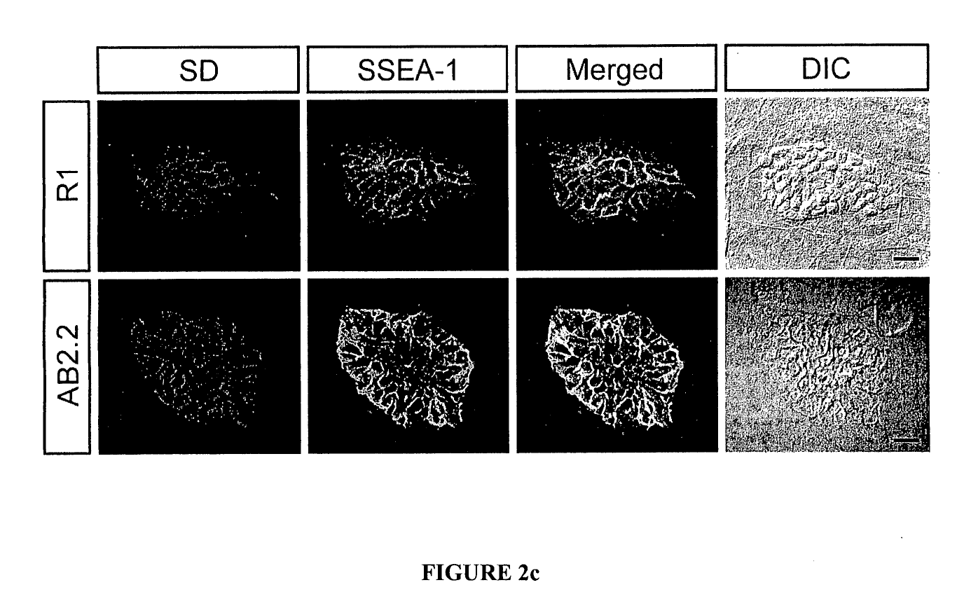

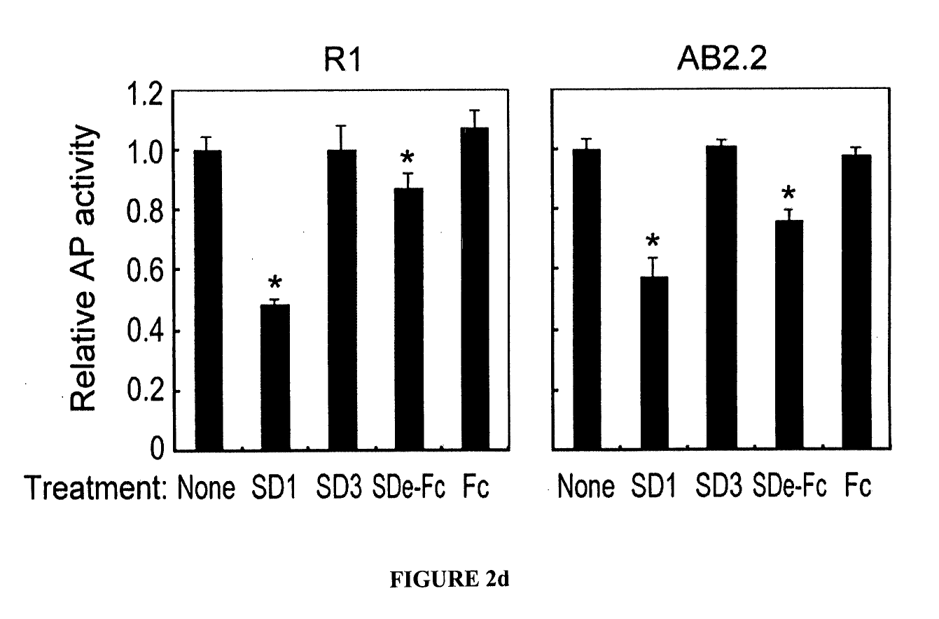

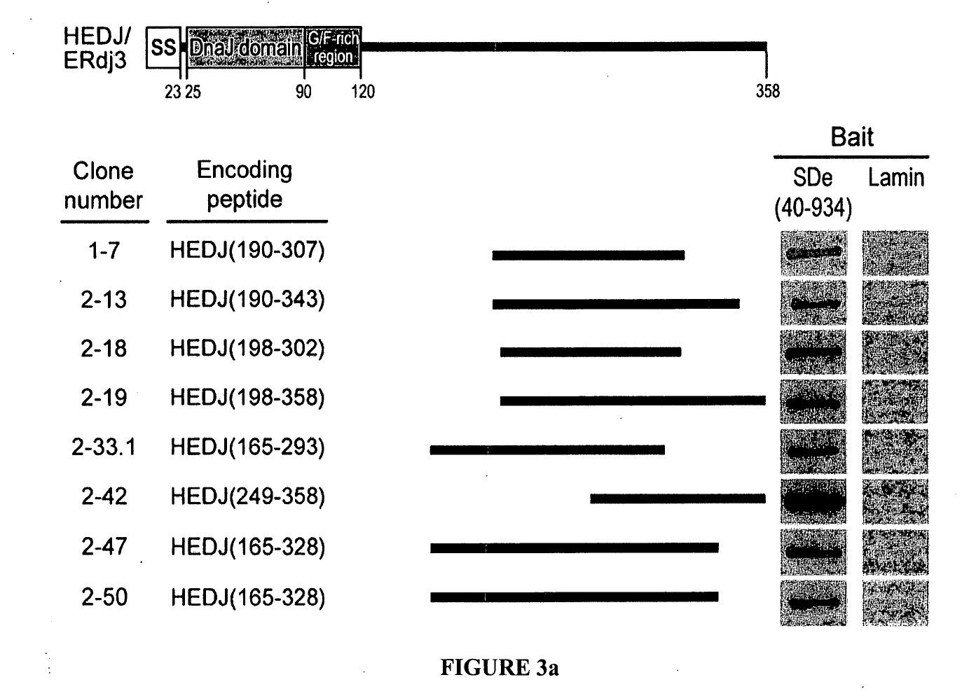

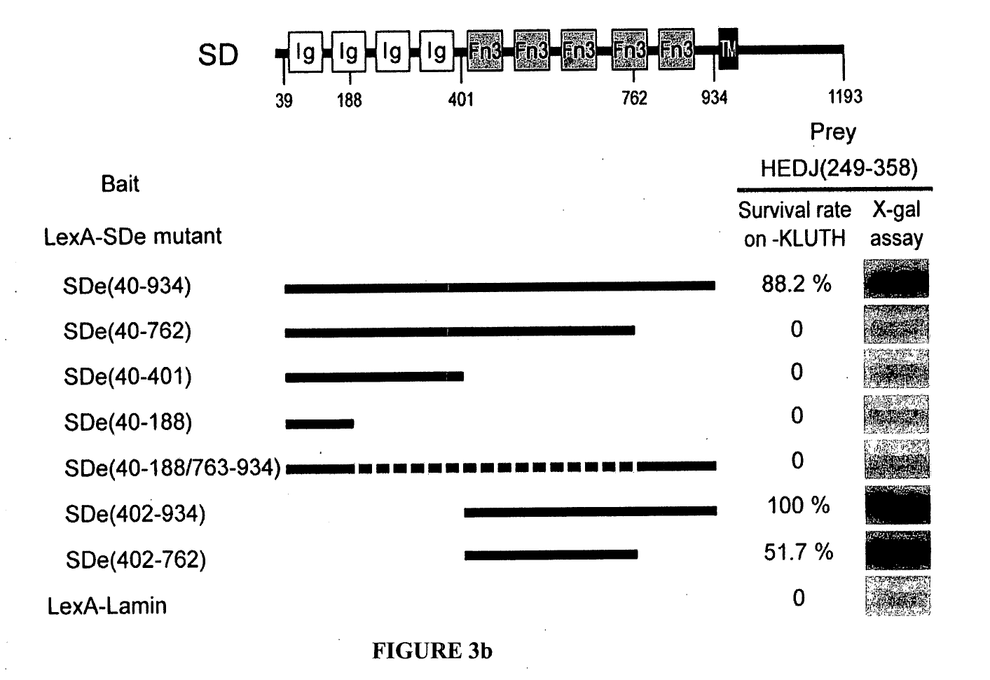

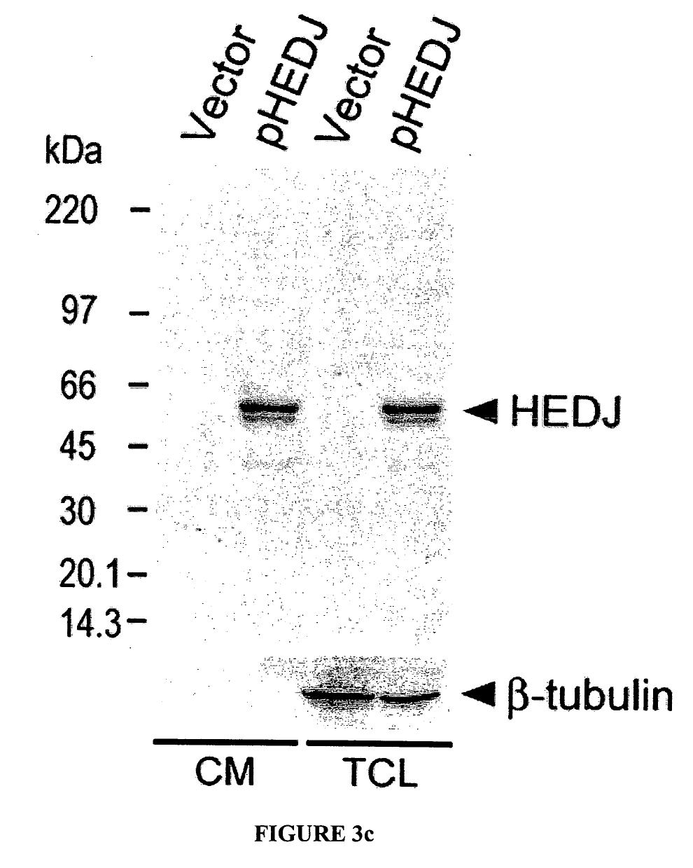

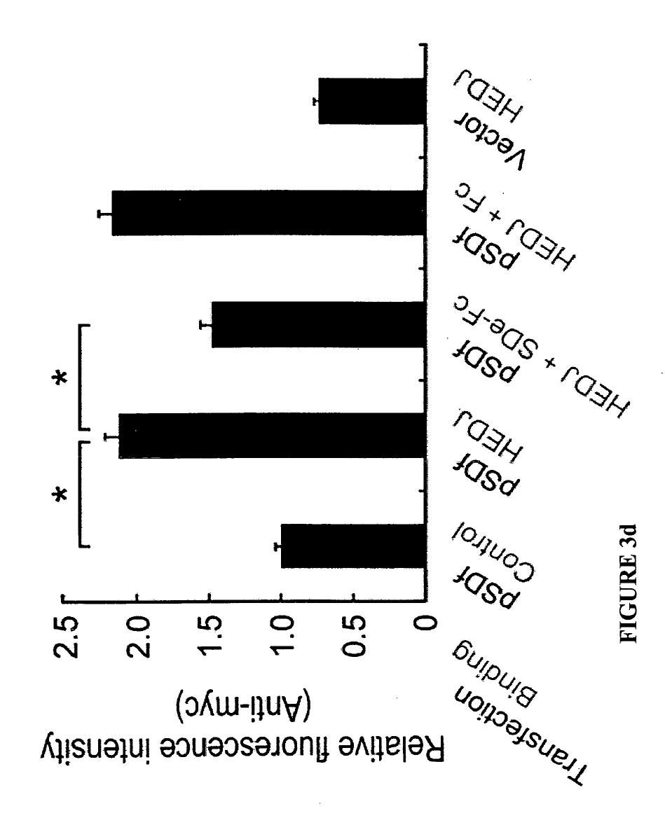

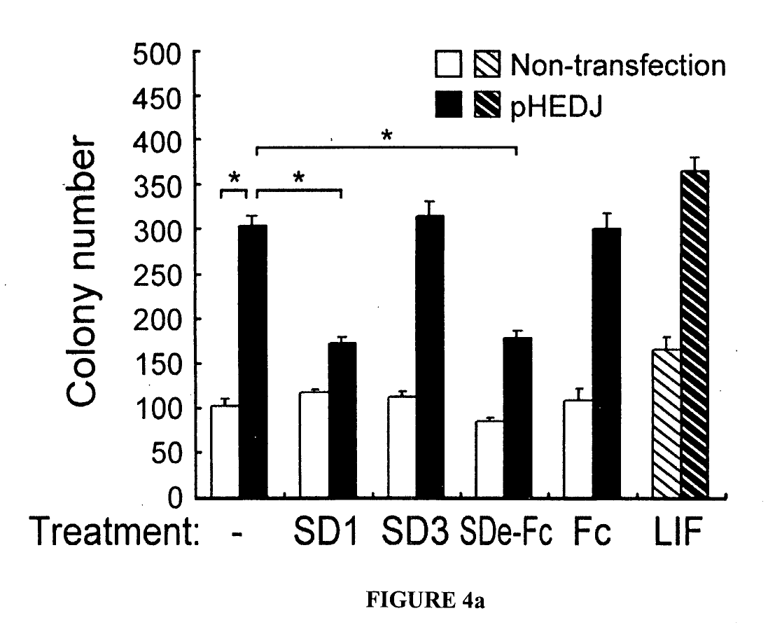

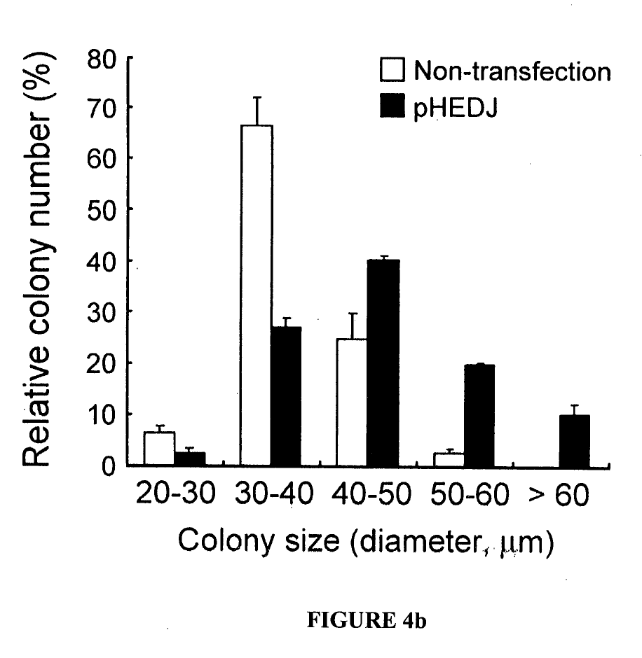

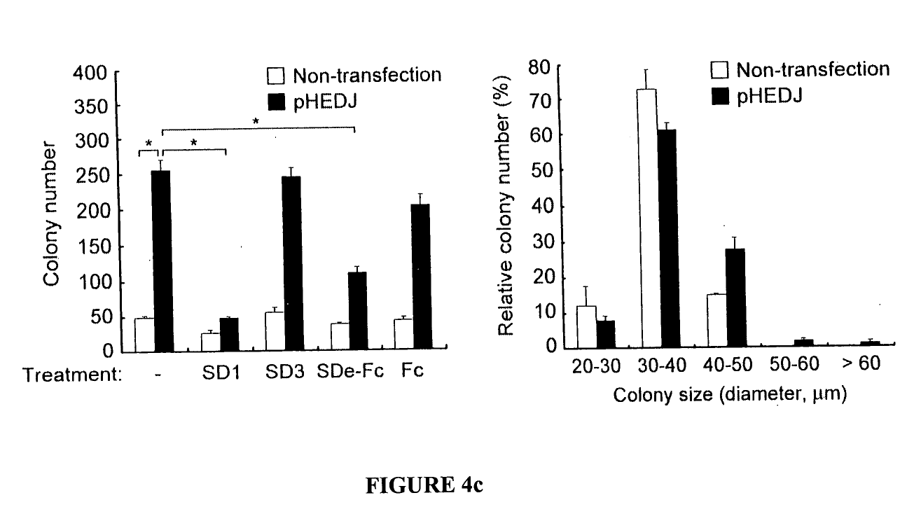

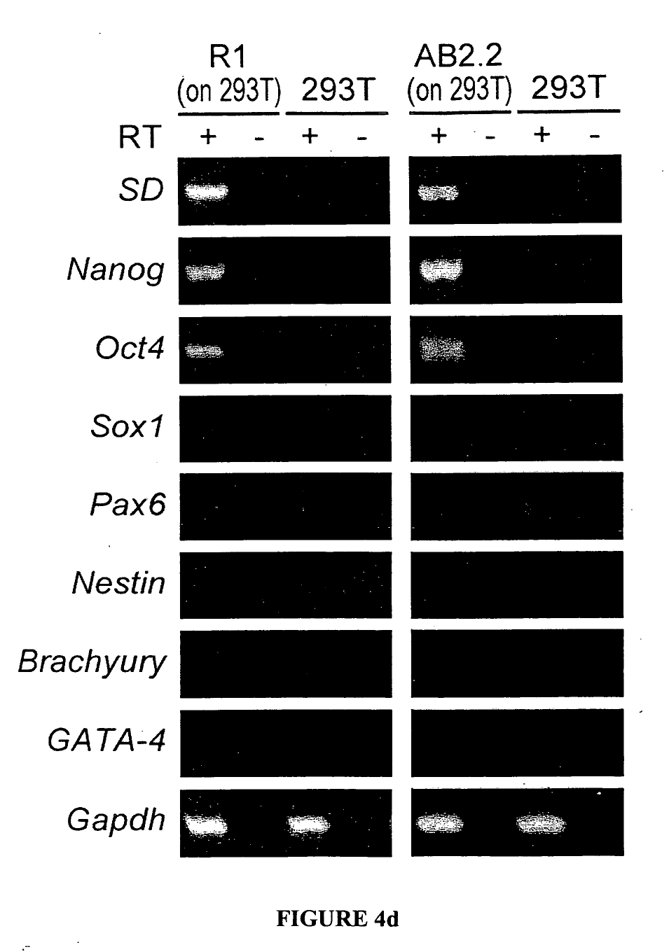

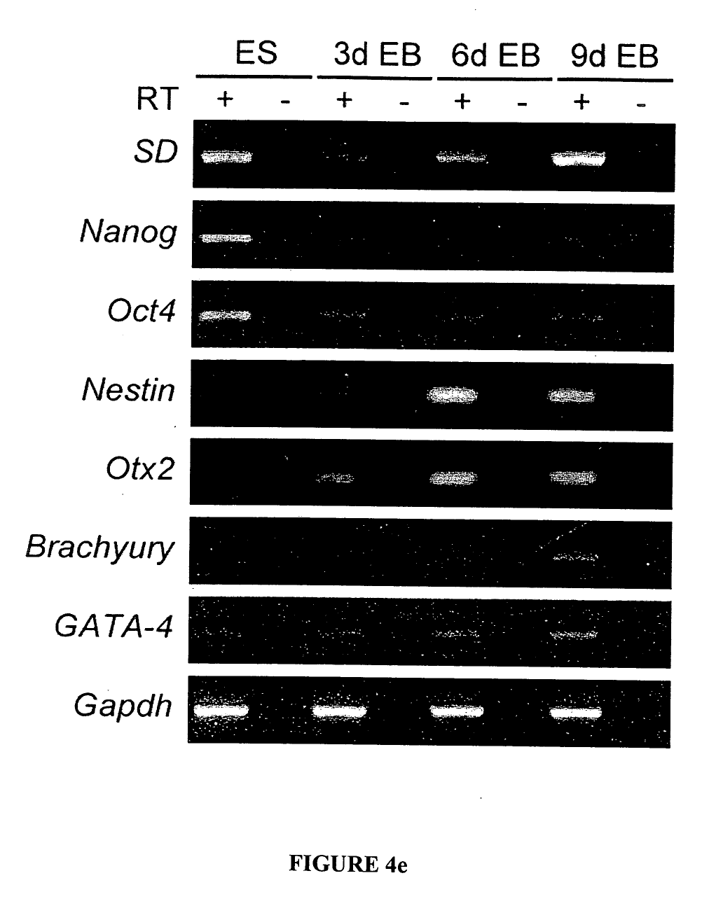

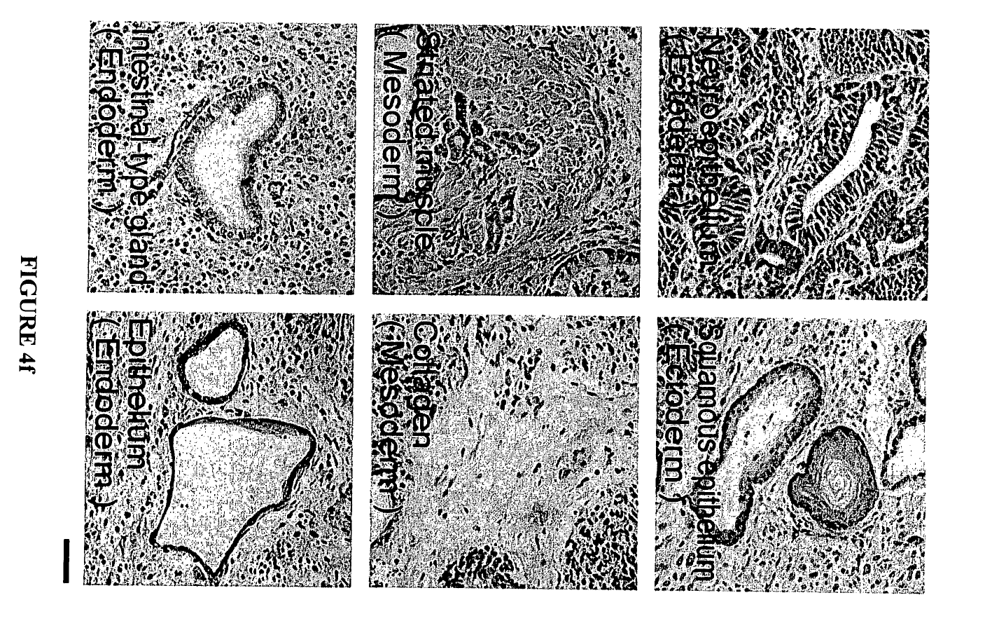

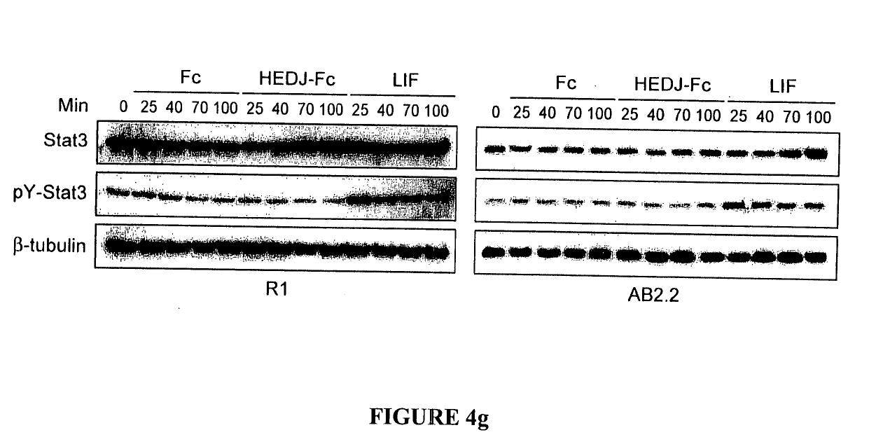

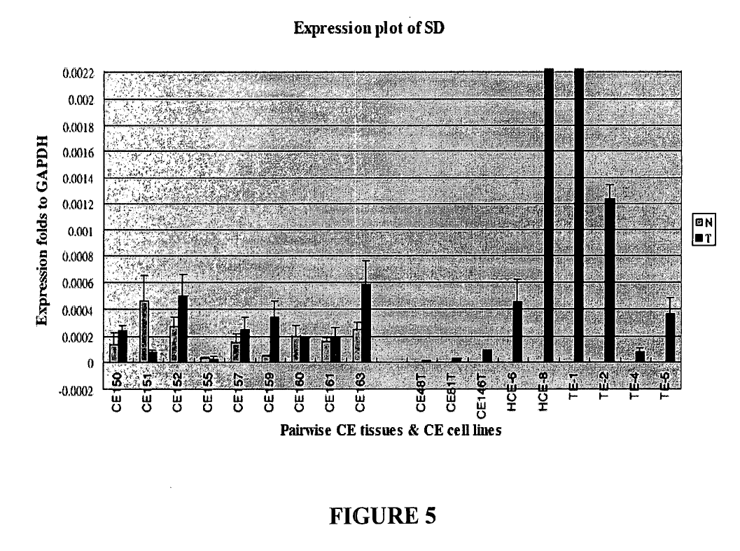

During an investigation of novel genes involved in early neural development by suppression subtractive hybridization¹¹, we identified partial DNA fragments of a novel gene, shen-dan (sd), which is expressed in rat embryonic day 10.5 (E10.5) neural tube but not in postnatal day 1 (P1) brain. Cloning of the rat sd sequence was achieved by 5′ rapid amplification of cDNA ends using E10.5 neural tube cDNA as the template. Sequences of the human and mouse sd genes were then acquired by database comparison and were confirmed by PCR. The deduced protein sequences of SD are highly conserved among these three species, with 86% identity between human and the two rodents, and 96% between mouse and rat (FIG. 1a). SD is predicted to be a transmembrane protein with four immunoglobulin (Ig) domains, five fibronectin type III (FnIII) repeats, eleven N-glycosylation sites, and a cytoplasmic tail that shares no significant homology with other proteins. We named this gene shen-dan (sd, Mandarin for elixir), inspired by its expression and effects on ES cells, as demonstrated here.

The most similar SD homologues in the Ig superfamily are Nope, Punc, Neogenin, and DCC (FIG. 1b). In addition, chromosomal locations of sd, nope, punc, and neogenin are all between 15q21 and 15q23. This strongly suggests that these genes are products of gene duplication. Expression of sd, like that of nope, punc, neogenin, and dcc¹³⁻¹⁵, is preferentially detected in the embryonic nervous system and is developmentally regulated. When analyzed by Northern blotting, expression of the rat sd transcripts, 8.7 kb and 4.2 kb, is high during neural tube formation (rat E10.5), gradually decreases in E12.5 and E14.5 brain, and disappears in the brain after E16.5 (FIG. 1c). Transcripts of sd are not detected in E14.5 embryonic non-neural tissues, nor in any P1 or adult rat tissues analyzed (data not shown). In situ hybridization analysis of E9 rat embryos, a period when the three germ layers have already formed, shows that sd is strongly expressed in mesoderm. Lower levels of sd can also be observed in ectoderm (FIG. 1d).

As Blast analysis of EST databases revealed that expression of sd occurs in mouse ES cells (library number ML. 10023), mouse ES cells were then used as a cell model to characterize effects of SD. We first examined expression of sd in two mouse ES cell lines, R1 and AB2.2, by RT-PCR. mRNA for sd is detected in R1 and AB2.2 (FIG. 2a), but not in the feeder layers for R1 and AB2.2, mouse embryonic fibroblasts (MEF) and STO cells. To determine whether SD expression in ES cells also occurs at the protein level, we generated antibodies against SD. Two His-tagged fusion proteins that contain either the extracellular domain (E peptide, rat SD amino acid 320 to 506) or the cytoplasmic tail (C peptide, rat SD amino acid 970 to 1193) were prepared in an E. coli expression system and purified by metal-chelating column. Monoclonal antibodies (mAb) against each of these fusion proteins were generated. In Western blots, SD1 mAb and SD2 mAb recognize the extracellular domain and the cytoplasmic tail, respectively (FIG. 2b). Two bands with apparent molecular weights of 186 kD and 174 kD bound both SD1 mAb and SD2 mAb when lysates of HEK293T cells transfected with pSDf (encoding the full length SD protein) are examined. The sizes of two bands are higher than anticipated, most likely due to glycosylation. In lysates of rat E10.5 embryo, however, only a faint 177-kD band is detected by both mAbs. Binding of mAbs to these bands is blocked by the appropriate peptides, showing the binding to be specific. The same bands are observed using another mAb, SD3, and a rabbit polyclonal antiserum, W4, both against the cytoplasmic tail (FIG. 2b and data not shown). SD2 mAb was then used to detect endogenous SD protein expression in R1 and AB2.2 cells. The staining of SD2 mAb is at the membranes of the R1 and AB2.2 cells, but is not present in the feeder cells (FIG. 2c). The pattern of SD expressed in ES cells is similar to that of SSEA-1¹⁶ (FIG. 2c). The cell membrane staining of SD was further confirmed using SD1 mAb to label live HEK293T cells overexpressing SDf.

It is possible that SD is involved in maintenance of ES cell pluripotency by acting as a receptor interacting with other factors. To evaluate this possibility, we added SD1 mAb in the medium to perturb SD function during the culturing of R1 and AB2.2 cells with feeder cells. The activity of alkaline phosphatase (AP), a marker for ES cells, was used as an index of ES phenotype. Strikingly, addition of SD1 mAb, but not SD3 mAb, lowers AP activity in individual ES cells. Overall, 40˜50% of the AP activity is inhibited by the presence of SD1 mAb during 3 day culture (FIG. 2d). A lower level of perturbation is also observed when the Fc-tagged SD extracellular domain (SDe-Fc) is present during the culture period. The results support the hypothesis that SD acts as a cell surface receptor.

To search for ligands of SD, a yeast two-hybrid approach was employed. The SD extracellular domain (SDe, rat SD amino acid 40 to 934) was used as a bait to screen an E7.5 mouse cDNA library. After analyzing 5.2 million clones, 54 partial DNA fragments were found to interact with SDe. Among these fragments, eight clones contained DNA sequences corresponding to the C-terminal domain of HEDJ/ERdj3 (FIG. 3a), a protein with a signal sequence and previously reported as an endoplasmic reticulum (ER)-resident chaperone¹⁷. To confirm the association of SD with HEDJ/ERdj3 and to delineate the interaction domains of SD with HEDJ/ERdj3, a series of sd deletion mutants were constructed, and their interaction with the C-terminal region of HEDJ/ERdj3 was evaluated by yeast two-hybrid assay. As shown in FIG. 3b, the FnIII motifs of the SD protein are critical for binding with HEDJ/ERdj3, especially the first three FnIII motifs.

It has previously been shown that the HEDJ/ERdj3 protein is present in the ER¹⁷. However, to act as a ligand of SD, it is necessary that the protein is secreted out of the cell. To determine whether HEDJ/ERdj3 is released from cells, HEK293T cells were transfected with pHEDJ that produces the C-terminal His-myc-tagged HEDJ/ERdj3 protein. As expected, the expression of HEDJ/ERdj3 was detected in the conditioned medium (CM) by a mAb against myc (9E10) (FIG. 3c). At least 50% of the total HEDJ/ERdj3 protein is secreted into the CM. The presence of this protein in the CM is not due to rupture of dying cells, as the cytosolic protein marker, β-tubulin, was not found in the CM. The secreted protein was then partially purified with a Ni⁺²-chelating column and subjected to the N-terminal amino acid sequencing. A peptide with the sequence of GRDFYKILGVPRSASIKDI (SEQ ID NO:4) was obtained, demonstrating that the secreted protein is HEDJ/ERdj3 and the predicted signal sequence has been deleted.

We next determined whether HEDJ/ERdj3 binds to SD protein in an in situ binding assay. Cells that had been transfected with pSDf were incubated with the CM containing HEDJ/ERdj3 for two hours before fixation with 4% paraformaldehyde, and then double-labeled with antibodies against SD (W4) and myc (9E10). Results from this analysis show that HEDJ/ERdj3 attaches to cells exhibiting SD on the cell surface but not to cells without SD expression. Moreover, 60% of the binding is abolished by adding 40 μg/ml of secreted Fc-tagged SDe protein during incubation (FIG. 3d). These findings, together with results from the yeast two-hybrid interaction, support HEDJ/ERdj3 as a ligand of SD.

The expression of hedj/erdj3 can be detected in a mouse 2-cell embryo EST library (ML. 1882) and is observed in R1, AB2.2 and their feeder cells (FIG. 2a), raising the possibility that HEDJ/ERdj3 is secreted from ES cells or feeder cells and, by binding to SD, may play a role in the maintenance of self-renewal of ES cells. To explore this possibility, we cultivated R1 cells at low density on pHEDJ-transfected HEK293T cells, which have neither endogenous HEDJ/ERdj3 nor LIF expression (FIG. 2a and data not shown). The number and size of ES colonies formed after 3 days on a cover glass (22×22 mm) were then quantified by counting SSEA-1-positive colonies. Approximate one hundred (103±8; mean±s.e.m.) of R1 colonies formed when grown on non-transfected HEK293T cells (FIG. 4a). Remarkably, the number of R1 colonies was increased by 200% when HEK293T cells overexpressing HEDJ/ERdj3 were used as the feeder layer. In contrast, LIF increases colony formation by only 65%. The size of the colonies also becomes larger in the presence of HEDJ/ERdj3 (FIG. 4b). These effects are mediated by the interaction between the secreted HEDJ/ERdj3 and the SD protein, as addition of either SD1 mAB (binds to SD protein) or SDe protein (binds to HEDJ/ERdj3) reduces the number of colonies induced by HEDJ/ERdj3 by 43.4% and 41.4%, respectively (FIG. 4a). Blocking of HEDJ/ERdj3 activity is not observed when SD3 mAb or Fc is used in the competition assay. Similar effects of HEDJ/ERdj3 on ES cell growth can also be demonstrated by using AB2.2 cells (FIG. 4c).

R1 and AB2.2 cells grown on HEDJ/ERdj3-expressing HEK293T cells display compact colonies of small cells with a large nuclear to cytoplasmic ratio, which is the typical morphology of ES cells. In addition, expression of several ES cell markers, including SSEA-1, Nanog, and Oct4, is preserved (FIG. 4d). These cells do not, however, express differentiation markers, such as Sox1, Pax-6, Nestin, Brachyury or GATA-4, until differentiation upon embryoid body formation (FIG. 4d, e). Moreover, HEDJ/ERdj3-cultured AB2.2 cells generate teratomas consisting of three germ-layer derivatives (FIG. 4f). Therefore, HEDJ/ERdj3 is able to maintain pluripotency of ES cells. Finally, treatment of R1 and AB2.2 cells with 25 μg/ml of partially purified Fc-tagged HEDJ/ERdj3 for 25 minutes or longer does not trigger phosphorylation of Stat3 (FIG. 4g), which is found following LIF addition, suggesting that the action of HEDJ/ERdj3 does not involve the LIF signaling pathway. Supporting this view, there is an additive effect on R1 clone formation when R1 cells are cultured in the presence of HEDJ/ERdj3 and LIF (FIG. 4a).

HEDJ/ERdj3, as well as four other Hsp40/DnaJ chaperones that localize in the ER lumen (ERdj1, ERdj2, ERdj4 and ERdj5), possess a N-terminal J domain that activates the ATPase activity of BiP (a Hsp70 chaperone) to facilitate protein folding¹⁷. HEDJ/ERdj 3, unlike other ERdjs, has no predicted transmembrane helix and no ER retrieval signal at the C-terminus, thus favoring its tendency of entering the secretory pathway¹⁸. Our observation that HEDJ/ERdj3 is present in CM corroborates this hypothesis. Therefore, HEDJ/ERdj3 is unique among ERdj family members, both in its cellular localization and in its biological functions. It acts as a chaperone activator when it resides in ER, but also acts as a ligand for SD protein when it is secreted into extracellular milieu. Although it is thought that the C-terminal domain of each ERdj family member forms a peptide-binding pocket to deliver polypeptide substrates to Hsp70 chaperones¹⁹, our finding that the C-terminal motif of HEDJ/ERdj3 binds to a specific protein and has effects other than those involved in protein folding is not without precedence. A recent study has shown that the SANT2 domain in the C-terminal region of ERdj 1 binds to α1-antichymotrypsin and antagonizes its inhibitory activity²⁰.

Maintaining ES cells in a pluripotent state in vitro requires the interaction of intrinsic factors and extrinsic instructive molecules²¹. Three transcription factors, Nanog, Oct4 and Stat3, act somewhat in parallel to maintain ES cells in a proliferative and undifferentiated state^{22, 23}. LIF and its related cytokines, by activating Stat3, are the only extrinsic proteins found so far to sustain pluripotency of ES cells in the presence of serum. However, LIF is not a necessary factor for the self-renewal of human ES cells²⁴ in vitro and lif-deficient mice develop normally¹⁵, revealing that LIF is not an indispensable factor for ES cells in vitro and inner cell mass of blastocysts in vivo. It is likely that there are other secreted molecules which can sustain the phenotypes of ES cells and the inner cell mass. The fact that a partially purified molecule is able to maintain growth of lif receptor-deficient ES cells also supports this view¹⁰. In this study, we uncovered in mouse ES cells a novel receptor, SD, and one of its ligands, HEDJ/ERdj3. The activation of SD by HEDJ/ERdj 3 stimulates growth of mouse ES colonies in a clonal-density experiment and maintains the stem cell phenotype.

An expression pattern analysis against the EST database shows that SD is also expressed in tumor cells, raising the possibility that SD is an oncogene. It is contemplated that SD mediates cell proliferation to facilitate tumor formation through its activation with HEDJ/ERdj3. Thus, we examined gene expression of SD in 9 pairs of esophageal carcinoma (CE, with normal and tumor tissue parts) and 9 esophageal carcinoma cell lines by quantitative PCR. A higher level of SD expression was observed in the tumor counterpart of three CE tumor pairs (CE152, CE159, CE163) and 5 CE cell lines (HCE-6, HCE-8, TE-1, TE-2, TE-5). These results (FIG. 5) thus support the role of SD and its ligand in tumor formation. Accordingly, inhibitor of SD or its ligand, particularly molecules that inhibit HEDJ/ERdj3 binding to SD, can be used to inhibit neoplastic cell proliferation and treat tumor. Furthermore, SD or HEDJ/ERdj3 can be used as targets in the screening and identification of anti-tumor drugs. For example, candidate drugs can be screened using a cell that expresses SD and proliferates in response to HEDJ/ERdj3. Alternatively, candidate drugs can be screened based on their activity in inhibiting the binding between SD and HEDJ/ERdj3 in an in vivo or in vitro assay.

Materials and Methods

Accession Numbers

Genbank databases accession numbers for human, mouse, and rat SD are AY630258, AY630257, and AY630256, respectively. The accession numbers for human, mouse, and rat HEDJ/ERdj3 are NP_{—}057390, XP_{—}148071, and AAQ91040.1, respectively. All these sequences are hereby incorporated by reference.

RT-PCR Analysis

Total cell RNA was prepared using the RNeasy Mini-Kit (Qiagen, Valencia, Calif., USA). Reverse transcription was performed using 3 μg of total RNA in a total volume of 20 μl with 100 U of superscript reverse transcriptase II (Gibco/BRL, Grand Island, N.Y., USA) at 50° C. for 1.5 hr. PCR was carried out in a GeneAmp 9700 (Applied Biosystems, Foster City, Calif., USA) for various cycles. Primers used and expected sizes are listed in Table 1.

Construction of Expression Vectors

The full-length or mutant SD fragments were amplified by PCR from E10.5 rat cDNA using high fidelity DNA polymerase (Roche) with various primers (Table 1) and were cut with proper restriction enzymes before being ligated into pEF1/Myc-His (Invitrogen) to give pSDf, pSDet, and pSDc. The full-length HEDJ/ERdj3 was amplified by PCR from E9 mouse cDNA and inserted in pEF1/Myc-His to give pHEDJ. To construct the yeast bait, the SDe fragment was ligated into pBTM116. Deletion constructs were derived from pBTM116-SDe by restriction enzyme digestion and in-frame ligation. Plasmids were verified by restriction enzyme mapping and sequencing. The Ig Fc region was first cloned into pEF1/Myc-His to give pFc. Secreted SDe was cloned into pFc after PCR. The secreted Fc and SDe-Fc fusion proteins were produced in HEK293T cells and purified on a protein A/G-Sepharose column (Oncogene, Boston, Mass., USA).

Immunoblotting and Fluorescence Staining

Mouse monoclonal anti-β-tubulin (E7), anti-SSEA-1 (IgM) (MC-480), and anti-myc (9E10) antibodies were from Developmental Studies Hybridoma Bank (University of Iowa, Iowa, USA). Rabbit polyclonal anti-STAT3 and anti-phospho-STAT3 antibodies were from Cell Signaling Technology (Beverly, Mass., USA). Cy3 labeled goat anti-mouse IgG1 antiserum was from Jackson ImmunoResearch Laboratories (West Grove, Pa., USA). FITC labeled goat anti-mouse IgG1 antiserum was from Chemicon International (Temecula, Calif., USA). FITC labeled goat anti-rabbit antiserum was from Zymed Laboratories (South San Francisco, Calif., USA). Other secondary antisera were from Bethyl Laboratories (Montgomery, Tex., USA). A standard protocol was used for the Western blotting and the membranes were developed with chemiluminescent ECL reagent (Amersham, Buckinghamshire, UK) according to manufacturer's protocols. Standard procedures were followed for immunostaining and fluorescence images were obtained by a fluorescence microscope (Nikon Eclipse TE300), equipped with a MicroMax cool CCD (Roper Scientific, Trenton, N.J., USA) or obtained by a laser scanning confocal microscope (Leica TCS NT or Olympus FV300). The digital images were processed using Adobe Photoshop software (San Jose, Calif., USA).

Alkaline Phosphatase Assay

R1 and AB2.2 cells were cultured on MEF and STO cells, respectively, in ES medium (DMEM with 15% FBS, 0.1 mM β-mercaptoethanol, 2 mM L-glutamine 100 U penicillin, and 100 μg/ml streptomycin) with or without 50 μg/ml of anti-SDe (SD1), anti-SDc (SD3) antibodies, or 40 μg/ml of purified SDe-Fc or Fc proteins. After 3-day culture, cells were fixed in 4% paraformaldehyde, and incubated for 10 min at 37° C. with 12 mM p-nitrophenyl phosphate which gives rise to a soluble product after alkaline phosphatase reaction. The supernatants were subjected to measurement of the OD at 405 nm. Cells were rinsed twice in PBS, once in alkaline phosphatase buffer, and stained for 10 min in the same buffer containing 12.75 μg/ml BCIP and 16.5 μg/ml NBT to produce an insoluble product at the cell surface.

In Situ Binding Assay

HEK293T cells were transfected with pSDf using FuGENE 6 (Roche). Sixty hours after transfection, 5 fold concentrated conditioned medium collected from pHEDJ transfected or non-transfected cells was added to HEK293T cells and incubated at room temperature for 2 hr. After washing once with PBS, the cells were fixed and immunofluorescence stained. Double staining of transfected SD and bound HEDJ was carried out using anti-SDc (W4) and anti-c-myc (9E10) antibodies and the appropriate secondary antisera. The fluorescence signal was quantified using ImageJ software (NIH, Bethesda, Md., USA).

Colony Forming Assay and Differentiation Assay

HEK293T cells were transiently transfected with pHEDJ using FuGENE 6. One day after transfection, the cells were pre-treated with 10 μg/ml mitomycin C at 37° C. for 3 hr, trypsinized, and plated on poly-L-lysine coated cover glasses (22×22 mm) at a density that ensured a confluent uniform monolayer after one more day culture. For co-culture, trypsinized ES cells were passed through a 70 μm mesh (Falcon, San Jose, Calif., USA), added to transfected HEK293T, and incubated for three days with fresh medium changed daily. All ES clones identified with SSEA-1 on a cover glass were photographed and numbers of colonies counted. For differentiation, AB2.2 cells were cultured for 4 passages with pHEDJ-transfected HEK293T cells. Cells were collected by trypsinization and plated on a 10-cm poly-L-lysine coated dishes for 30 min twice to remove HEK293T cells. The HEDJ-cultured AB2.2 cells were grown on STO cells for another 2˜3 passages to gain sufficient cells. For embryoid body formation, ES cells were digested with trypsin, collected in medium and plated 1 hr to allow the STO cells to adhere. Non-adherent cells were collected and plated in a 9-cm bacterial Petri dish at 15000 cells per 7 ml. Embryoid bodies were collected on different days and analyzed by RT-PCR. For teratoma formation, approximately 2×10⁶ HEDJ-cultured AB2.2 cells were injected subcutaneously into nude mice. After 4-5 weeks, teratoma was dissected, fixed with 4% paraformaldehyde, and processed for histological examination with hematoxylin and eosin staining.

### REFERENCES

1. Kim, J. H. et al. Dopamine neurons derived from embryonic stem cells function in an animal model of Parkinson's disease. Nature 418, 50-56 (2002).
2. Hori, Y. et al. Growth inhibitors promote differentiation of insulin-producing tissue from embryonic stem cells. Proc Natl Acad Sci USA 99, 16105-16110 (2002).
3. Wichterle, H., Lieberam, I., Porter, J. A. and Jessel, T. M. Directed differentiation of embryonic stem cells into motor neurons. Cell 110, 385-397 (2002).
4. Geijsen, N. et al. Derivation of embryonic germ cells and male gametes from embryonic stem cells. Nature 247, 148-154 (2004).
5. Sato, N., L. M., Skaltsounis, L., Greengard, P. and Brivanlou, A. H. Maintenance of pluripotency in human and mouse embryonic stem cells through activation of Wnt signaling by a pharmacological GSK-3-specific inhibitor. Nature Med. 10, 55-63 (2004).
6. Qi, X. et al. BMP4 supports self-renewal of embryonic stem cells by inhibiting mitogen-activated protein kinase pathways. Proc Natl Acad Sci USA 101, 6027-6032 (2004).
7. Smith, A. G. et al. Inhibition of pluripotential embryonic stem cell differentiation by purified polypeptides. Nature 336, 688-690 (1988).
8. Williams, R. L. et al. Myeloid leukaemia inhibitory factor maintains the developmental potential of embryonic stem cells. Nature 336, 684-687 (1988).
9. Ying, Q. L., Nichols, J., Chambers, I. and Smith, A. BMP induction of Id proteins suppresses differentiation and sustains embryonic stem cell self-renewal in collaboration with STAT3. Cell 115, 282-292 (2003).
10. Dani, C. et al. Paracrine induction of stem cell renewal by LIF-deficient cells: a new ES cell regulatory pathway. Dev. Biol. 203, 149-162 (1998).
11. Diatchenko, L. et al. Suppression subtractive hybridization: a method for generating differentially regulated or tissue-specific cDNA probes and libraries. Proc. Natl. Acad. Sci. USA 93, 6025-6030 (1996).
12. Kappen, C. and Salbaum, J. M. 09/15: comparative genomics of a conserved chromosomal region associated with a complex human phenotype. Genomics 73, 171-178 (2001).
13. Salbaum, J. M. Punc, a novel mouse gene of the immunoglobulin superfamily, is expressed predominantly in the developing nervous system. Mech. Dev. 71, 201-204 (1998).
14. Salbaum, J. M. and Kappen, C. Cloning and expression of Nope, a new mouse gene of the immunoglobulin superfamily related to guidance receptors. Genomics 64, 15-23 (2000).
15. Gad, J. M., Keeling, S. L., Wilks, A. F., Tan, S. S. and Cooper, H. M. The expression patterns of guidance receptors, DCC and Neogenin, are spatially and temporally distinct throughout mouse embryogenesis. Dev. Biol. 192, 258-273 (1997).
16. Solter, D. and Knowles, B. B. Monoclonal antibody defining a stage-specific mouse embryonic antigen (SSEA-1). Proc. Natl. Acad. Sci. USA 75, 5565-5569 (1978).
17. Yu, M., Haslam, R. H. A. and Haslam, D. B. HEDJ, an Hsp40 co-chaperone localized to the endoplasmic reticulum of human cells. J. Biol. Chem. 275, 24984-24992 (2000).
18. Ohtsuka, K. and Hata, M. Mammalian HSP40/DNAJ homologs: cloning of novel cDNAs and a proposal for their classification and nomenclature. Cell Stress & Chaperones 5, 98-112 (2000).
19. Fewell, S. W., Travers, K. J., Weissman, J. S. and Brodsky, J. L. The action of molecular chaperones in the early secretory pathway. Annu. Rev. Genet. 35, 149-191 (2001).
20. Kroczynska, B., Evangelista, C. M., Samant, S. S., Elguindi, E. C. and Blond, S. Y. The SANT2 domain of the murine tumor cell DnaJ-like protein 1 human homologue interacts with alpha 1-antichymotrypsin and kinetically interferes with its serpin inhibitory activity. J. Biol. Chem. 279, 11432-11443 (2004).
21. Smith, A. G. Embryo-derived stem cells: of mice and men. Annu. Rev. Cell Dev. Biol. 17, 435-462 (2001).
22. Chambers, I. et al. Functional expression cloning of Nanog, a pluripotency sustaining factor in embryonic stem cells. Cell 113, 643-655 (2003).
23. Mitsui, K. et al. The homeoprotein Nanog is required for maintenance of pluripotency in mouse epiblast and ES cells. Cell 113, 631-642 (2003).
24. Reubinoff, B. E., Pera, M. F., Fong, C. Y., Trounson, A. and Bongso, A. Embryonic stem cell lines from human blastocysts: somatic differentiation in vitro. Nat. Biotechnol. 18, 399-404 (2000).
25. Stewart, C. L. et al. Blastocyst implantation depends on maternal expression of leukemia inhibitory factor. Nature 359, 76-79 (1992).

All of the publications, patents and patent applications cited above or elsewhere in this application are herein incorporated by reference in their entirety to the same extent as if the disclosure of each individual publication, patent application or patent was specifically and individually indicated to be incorporated by reference in its entirety.

TABLE 1

Primers used in this study.

| | | |
|---|---|---|
| Gene Name | Primers (Forward and Reverse) | Product Size (bp) |

| | | |
|---|---|---|
| Brachyury | (F) 5′-CTATGCTCATCGGAACAG-3′ (SEQ ID NO:5) | 419 |
| | (R) 5′-GTGACACAGGTGTCCACG-3′ (SEQ ID NO:6) | |
| Fc | (F) 5′-GCAAGCGGCCGCGCAGAGCCCAAATCTTGTGAC-3′ (Not I) (SEQ ID NO:7) | 724 |
| | (R) 5′-GCAATCTAGACTATTTACCCGGAGACAGGG-3′ (Xba I) (SEQ ID NO:8) | |
| Gapdh | (F) 5′-ACCACAGTCCATGCCATCAC-3′ (SEQ ID NO:9) | 452 |
| | (R) 5′-TCCACCACCCTGTTGCTGTA-3′ (SEQ ID NO:10) | |
| GATA-4 | (F) 5′-GCCTCTACATGAAGCTCC-3′ (SEQ ID NO:11) | 421 |
| | (R) 5′-CCATGACTGTCAGCCAGG-3′ (SEQ ID NO:12) | |
| HEDJ/ERdj3 | (F) 5′-GCAAGGATCCGCTGAGAGGGTAGGACCAGG-3′ (BamH I) (SEQ ID NO:13) | 1130 |
| | (R) 5′-GCAAGAATTCATAGCCCTGCAGCCCGTT-3′ (EcoR I) (SEQ ID NO:14) | |
| Nanog | (F) 5′-GAGTGTGGGTCTTCCTGGTC-3′ (SEQ ID NO:15) | 912 |
| | (R) 5′-CCTGGTGGAGTCACAGAGTA-3′ (SEQ ID NO:16) | |
| Nestin | (F) 5′-CCCTTGGATTAGAGGCTG-3′ (SEQ ID NO:17) | 525 |
| | (R) 5′-CTGGCTCATCTTCTACTC-3′ (SEQ ID NO:18) | |
| Oct4 | (F) 5′-CCCCCACTTCACCACACTC-3′ (SEQ ID NO:19) | 249 |
| | (R) 5′-GCATCACTGAGCTTCTTTCCC-3′ (SEQ ID NO:20) | |
| Otx2 | (F) 5′-GGTTTGGTTCAAGAATCG-3′ (SEQ ID NO:21) | 483 |
| | (R) 5′-GCTGCTTTGCTTTCAGTC-3′ (SEQ ID NO:22) | |
| Pax6 | (F) 5′-TGAAGCGGAAGCTGCAAAGA-3′ (SEQ ID NO:23) | 173 |
| | (R) 5′-GGCCCTTCGATTAGAAAACC-3′ (SEQ ID NO:24) | |
| SD | (F) 5′-GCCAAGCTGCTTCATTGATGC-3′ (SEQ ID NO:25) | 442 |
| | (R) 5′-GTCTCCATGCTTCTTCGGTGC-3′ (SEQ ID NO:26) | |
| SDc | (F) 5′-GCAAACTAGTCATGGCCAGGAAGTCATCCGCCTCTAAG-3′ (Spe I) (SEQ ID NO:27) | 698 |
| | (R) 5′-GCAAGCGGCCGCGACGGGAACACTCTCGGC-3′ (Not I) (SEQ ID NO:28) | |
| SDe | (F) 5′-GCAAGTCGACAGGACGCAACGGTCACA-3′ (Sal I) (SEQ ID NO:29) | 2707 |
| | (R) 5′-GCAAGTCGACCTAGCCTGAATAAACTTTGGC-3′ (Sal I) (SEQ ID NO:30) | |
| SDet | (F) 5′-GCAAGCGGCCGCCATGGCGCCTCCCGTGCGC-3′ (Not I) (SEQ ID NO:31) | 2966 |
| | (R) 5′-GCAAGCGGCCGCTAGTAATCTGGAACATCGTATGGGTACTTCCTGGCTTTGCT | |
| | TCG-3′ (Not I) (SEQ ID NO:32) | |
| SDf | (F) 5′-GCAAGCGGCCGCCATGGCGCCTCCCGTGCGC-3′ (Not I) (SEQ ID NO:33) | 3603 |
| | (R) 5′-GCAAGCGGCCGCGACGGGAACACTCTCGGC-3′ (Not I) (SEQ ID NO:34) | |
| Sox1 | (F) 5′-TTACTTCCCGCCAGCTCTTC-3′ (SEQ ID NO:35) | 369 |
| | (R) 5′-GCATTTTGGGGGTATCTCTC-3′ (SEQ ID NO:36) | |

1. A method for proliferating embryonic stem cells, comprising contacting at least one embryonic stem (ES) cell with an effective amount of HEDJ/ERdj3 to result in enhanced proliferation of the ES cell.

2. The method of claim 1 wherein the ES cell is a mouse ES cell.

3. The method of claim 1 wherein the ES cell is a human ES cell.

4. The method of claim 1 wherein the HEDJ/ERdj3 is provided by a feeder cell that expresses the HEDJ/ERdj3.

5. A method of enhancing differentiation of embryonic stem (ES) cells, comprising contacting at least one ES cell with an effective amount of an antagonist for SD.

6. The method of claim 5 wherein the antagonist is an antibody.

7. The method of claim 6 wherein the antibody recognizes SD or HEDJ/ERdj3.

8. The method of claim 5 wherein the antagonist comprises the region of SD that contains five fibronectin type III repeats.

9. The method of claim 5 wherein the antagonist comprises the extracellular domain of SD.

10. The method of claim 5 wherein the antagonist consists of the extracellular domain of SD.

11. The method of claim 5 wherein the ES cell is a mouse ES cell.

12. The method of claim 5 wherein the ES cell is a human ES cell.

13-16. (canceled)

17. A method of screening for agents that enhance ES cell proliferation, comprising:
(a) determining the activity of a test agent to bind to SD and identifying the agents that bind SD as potential agents; and
(b) determining the activity of the potential agents to enhance ES cell proliferation.

18-36. (canceled)
